(19) United States
(12) Patent Application Publication
(10) Pub. No.: US 2012/0150155 A1
(43) Pub. Date: Jun. 14, 2012

(54) Instrument Control Device

(75) Inventors: Gary KAPPEL, Acton, MA (US); Larry Stanton, Burlington, MA (US)

(73) Assignee: Boston Scientific Scimed, Inc.

(21) Appl. No.: 13/304,916

(22) Filed: Nov. 28, 2011

Related U.S. Application Data
(60) Provisional application No. 61/421,955, filed on Dec. 10, 2010.

(51) Int. Cl. A61B 17/00

(52) U.S. Cl. 606/1

(57) ABSTRACT

Embodiments include an instrument including an end effector located near a distal end of the instrument, an elongated member connected to the end effector, and a handle located near a proximal end of the instrument. The instrument also includes a ball and socket joint including a ball rotatable in a socket. The ball and socket joint connects the elongated member to the handle, and the handle is configured to move with respect to the elongated member via the ball and socket joint. The instrument further includes a control member connected to one of the ball or the socket, and the control member extends through the elongated member and connects the ball and socket joint to the end effector to control movement of the end effector.

This application claims the benefit of priority from U.S. Provisional Application No. 61/421,955, filed Dec. 10, 2010, which is herein incorporated by reference in its entirety.

### FIELD

Embodiments of the invention include medical instruments and more particularly medical instruments including control devices and related methods of use.

### BACKGROUND

Minimally invasive surgical instruments, such as endoscopic and laparoscopic devices, can provide access to surgical sites while minimizing patient trauma. Although the growing capabilities of such therapeutic and diagnostic devices allow physicians to perform an increasing variety of surgeries through traditional minimally invasive routes, further refinements may allow surgical access through even less invasive routes. Currently some robotic systems and other complex systems have been proposed to allow surgical access via a natural orifice. The user interface is remote from surgical instruments and/or end effectors. Unfortunately, these systems are generally large, expensive, and complicated. In addition, they fail to provide the tactile user feedback which traditional devices can provide. Accordingly, there is room for further refinement to conventional minimally invasive surgical devices and a need to develop new surgical systems.

It is to be understood that both the foregoing general description and the following detailed description are exemplary and explanatory only and are not restrictive of the invention.

### SUMMARY

According to an embodiment, an instrument includes an end effector located near a distal end of the instrument, an elongated member connected to the end effector, and a handle located near a proximal end of the instrument. The instrument also includes a ball and socket joint including a ball rotatable in a socket. The ball and socket joint connects the elongated member to the handle, and the handle is configured to move with respect to the elongated member via the ball and socket joint. The instrument further includes a control member connected to one of the ball or the socket, and the control member extends through the elongated member and connects the ball and socket joint to the end effector to control movement of the end effector.

According to another embodiment, an instrument includes an end effector located near a distal end of the instrument, an elongated member connected to the end effector, and a handle located near a proximal end of the instrument. The handle includes a shaft and a movable member that is movable with respect to the shaft. The instrument also includes a ball and socket joint connecting the elongated member to the handle, and the handle is configured to move with respect to the elongated member via the ball and socket joint. The instrument further includes a control member connected to the movable member and the end effector. The control member extends through the ball and socket joint and the elongated member, and the movable member is configured to move with respect to the shaft to control the end effector via the control member.

According to a further embodiment, a method for controlling an instrument includes controlling an end effector near a distal end of an instrument by pivoting a handle with respect to an elongated member of the instrument via a ball and socket joint of the instrument to control a first control member connecting the ball and socket joint to the end effector. The first control member is connected to one of a ball or a socket of the ball and socket joint. The end effector is also controlled by sliding a movable member of the handle of the instrument with respect to a shaft of the handle to control a second control member connecting the movable member to the end effector.

Additional objects and advantages of the invention will be set forth in part in the description which follows, and in part will be obvious from the description, or may be learned by practice of the invention. The objects and advantages of the invention will be realized and attained by means of the elements and combinations particularly pointed out below.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a proximal end of an endoscopy system including an instrument, according to an exemplary embodiment;

FIG. 2 is a perspective view of a distal end of the endoscopy system of FIG. 1;

FIG. 3 is a perspective view of a proximal end of the instrument of FIG. 1 in one configuration;

FIG. 4 is a perspective view of a proximal end of the instrument of FIG. 1 in another configuration;

FIG. 5 is a cross-sectional view of a ball of a ball and socket joint of the instrument of FIG. 1;

FIG. 6 is a top view of a handle portion of an instrument, according to another exemplary embodiment;

FIG. 7 is a cross-sectional side view of a locking mechanism for locking an instrument with respect to an elongated member, according to an exemplary embodiment;

FIG. 8 is a cross-sectional side view of a locking mechanism for locking an instrument with respect to an elongated member, according to another exemplary embodiment; and

FIG. 9 is a cross-sectional side view of a braking mechanism for assisting in positioning an instrument with respect to an elongated member, according to an exemplary embodiment.

### DESCRIPTION OF THE EMBODIMENTS

Reference will now be made in detail to exemplary embodiments of the invention, examples of which are illustrated in the accompanying drawings. Wherever possible, the same reference numbers will be used throughout the drawings to refer to the same or like parts.

The terms “proximal” and “distal” are used herein to refer to the relative positions of the components of the exemplary endoscopy system 10. When used herein, “proximal” refers to a position relatively closer to the exterior of the body or closer to the surgeon using the endoscopy system 10. In contrast, “distal” refers to a position relatively further away from the surgeon using the endoscopy system 10 or closer to the interior of the body.

In addition, while the discussion of systems and methods below may generally refer to “surgical instruments,” “surgery,” or a “surgical site” for convenience, the described systems and their methods of use are not limited to tissue resection and/or repair. In particular, the described systems may be used for inspection and diagnosis in addition, or as an alternative, to surgical treatment. The treatment is not limited to any particular treatment. Various other exemplary treatment devices and methods are referred to herein. Moreover, the systems described herein may perform non-medical applications such as in the inspection and/or repair of machinery.

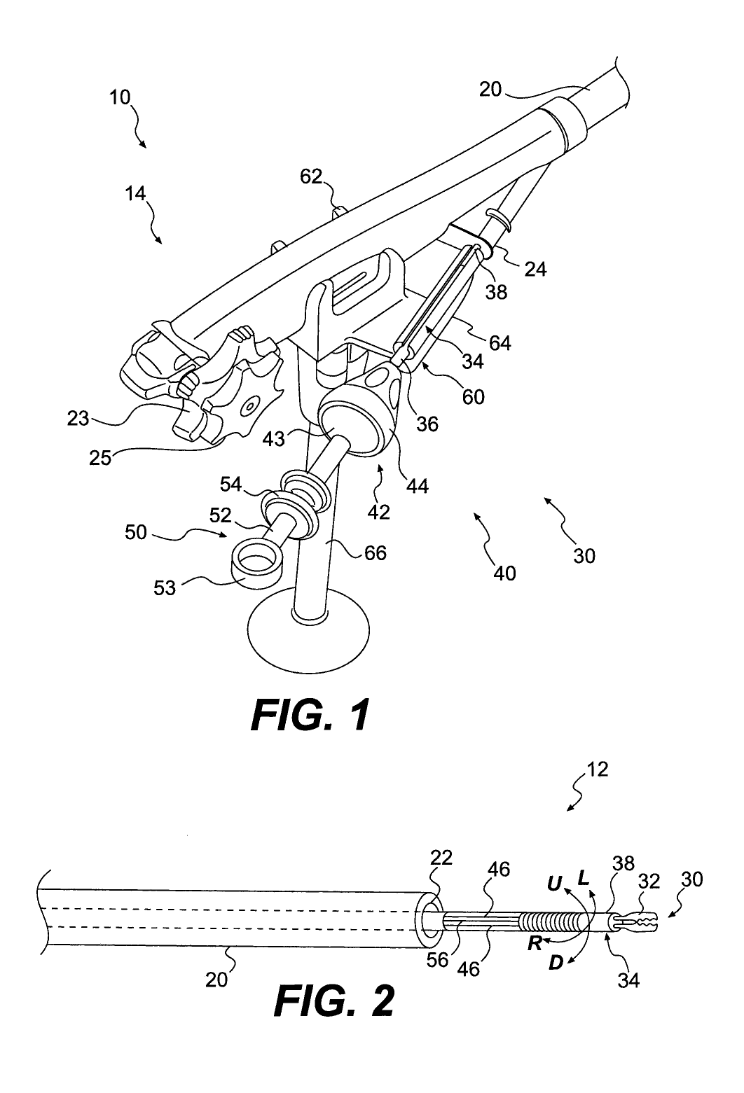

FIGS. 1-5 depict an exemplary endoscopy system 10 that may be used for any therapeutic or diagnostic endoscopic procedure and the components thereof. The phrase “endoscopic procedure” is broadly used to indicate any medical procedure that may be performed by inserting an endoscope, guide tube, catheter, or any other medical device into the body through any anatomic opening. The endoscopy system 10 may be used for performing surgery at a relative distance via medical instruments including or directly connected to user controls. The endoscopy system 10 may be adapted for, but not limited to, trans-oral, trans-anal, trans-vaginal, trans-urethral, trans-nasal, trans-cranial, transluminal, laparoscopic, thorascopic, orthopedic, through the ear, and/or percutaneous access. The components of the endoscopy system 10 described below may be made of any suitable material capable of being inserted into the body, e.g., a suitable biocompatible material.

As shown in FIGS. 1 and 2, the endoscopy system 10 may include an elongated member 20 including one or more channels 22 (FIG. 2). The channel 22 may extend substantially longitudinally (axially) within the elongated member 20, and generally between a distal end and a proximal end of the elongated member 20. The elongated member 20 may be configured to be advanced through any body cavity or body lumen of a patient. The elongated member 20 may be flexible, for example, to be able to traverse tortuous anatomy. One end of the elongated member 20 may be positioned near a distal end 12 of the endoscopy system 10, as shown in FIG. 2, and this end of the elongated member 20 may be positioned internal to the body. An opposite end of the elongated member 20 may be positioned near a proximal end 14 of the endoscopy system 10, as shown in FIG. 1, and this end of the elongated member 20 may be positioned external to the body. The elongated member 20 may include imaging and light capabilities, and may also include structure for steering a distal end of the elongated member 20. That structure may include rotatable control knobs 23, 25 positioned at a proximal handle of the elongated member 20. The knobs 23, 25 may connect the control wires or cables (not shown) within the elongated member 20, to provide up/down and left/right steering of the distal end of the elongated member 20. The elongated member 20 may also include an adapter for allowing delivery of electrical and/or radiofrequency energy to the distal end of the elongated member 20.

One or more instruments 30 may be inserted through the channel 22 in the elongated member 20. For example, as shown in FIG. 1, the elongated member 20 may be an endoscope, colonscope, or other type of guide tube that includes one or more ports 24 that opens to the channel 22. The instrument 30 may be slidably inserted through the port 24 to enter the channel 22. As shown in FIG. 1, the port 24 may be provided at an angle to the channel 22 in the elongated member 20.

To position the elongated member 20 and the instrument 30, the distal end of the elongated member 20 may be inserted first into an opening in the patient and advanced into the patient (e.g., advanced into a body organ, through a body lumen, etc.), and then the instrument 30 may be advanced through the port 24 and the channel 22 of the elongated member 20 into the patient so that the instrument 30 may be used during an endoscopic procedure. Alternatively, the instrument 30 may be advanced through the elongated member 20 before the elongated member 20 is inserted into the opening in the patient. The port 24 may have a seal for preventing inflow and/or outflow of fluids from and/or to the patient's body via the channel 22.

As shown in FIG. 2, the instrument 30 may include an end effector 32 attached to a distal end of an elongated member 34 (a portion of which is shown cut away in FIG. 2 to show cables 46, 56 inside the elongated member 34). The end effector 32 may include a device configured to assist in performing an endoscopic or surgical procedure. For example, the end effector 32 may include, but is not limited to, a cutting device (with or without power) (e.g., scissors, tissue cutter, etc.), forceps, a fixation device, a manipulation device, a dissection device, a support device, a sealing device, a needle holder, a closure device (e.g., clips, staples, loops, ligator, suturing device, etc.), a retrieval device (e.g., snare, basket, loop, a fluid extraction device, etc.), a tissue exploration device (e.g., optical device, illumination device, etc.), a tissue sampling or biopsy device, any device with pivoting jaws, a delivery device, a device for aiding in the patency of a lumen or for dilating an opening (e.g., a balloon or other expandable member, stent, wire structure, etc.), a grasping device, a tissue retractor or spacer, etc. Accordingly, the instrument 30 and the end effector 32 may be any type of suitable instrument and end effector known to those skilled in the art. Some exemplary configurations of elongated members and instruments are disclosed, for example, in U.S. Patent Application Publication No. 2008/0188890, entitled “Multi-Part Instrument Systems and Methods” and U.S. Patent Application Publication No. 2008/0188868, entitled “Direct Drive Endoscopy Systems and Methods,” each of which is hereby incorporated by reference in its entirety. In some embodiments, multiple end effectors 32 may be located at the distal end of the elongated member 34, and/or multiple instruments 30 including respective end effectors 32 may be inserted through the channel(s) 22 in the elongated member 20.

The instrument 30 may be bent or articulated into a desired configuration to perform a procedure. The instrument 30 may be flexible, rigid, bendable, straight, malleable, etc., and may include sections of different degrees of flexibility/rigidity. For example, as shown in FIGS. 1, 3, and 4, the elongated member 34 of the instrument 30 may include a proximal portion 36 and a distal portion 38. The distal portion 38 may be relatively flexible and/or more flexible than the proximal portion 36 to allow the instrument 30 to be slidably inserted through the channel 22 and the port 24 in the elongated member 34. The flexible distal portion 38 allows the instrument 30 to pass through passageways that are not straight, such as the connection between the port 24 and the channel 22 shown in FIG. 1 where the port 24 is located at an angle from the channel 22. The relatively flexible distal portion 38 may be, e.g., approximately 3 to 6 feet long.

The proximal portion 36 may be relatively rigid and/or more rigid than the distal portion 38 to allow the proximal portion 36 to be slidably received in an instrument bracket 64 (FIG. 1) or other supporting device as described below. The relatively rigid proximal portion 36 may be formed separately from the relatively flexible distal portion 38, and then the proximal portion 36 and the distal portion 38 may be connected together. Alternatively, the elongated member 34 may be substantially entirely relatively flexible, and the proximal portion 36 may be modified to be relatively rigid compared to the distal portion 38. For example, a relatively rigid tube may be slipped over the proximal portion 36 in order to make the proximal portion 36 relatively rigid. As another example, the proximal portion 36 may be reinforced by a relatively rigid material in order to make the proximal portion 36 relatively rigid.

The instrument bracket 64 may include a slot or surface, e.g., with generally U-shaped cross-section, to slidably receive and support the elongated member 34 of the instrument 30, such as the proximal portion 36 of the elongated member 34. For example, the relatively rigid proximal portion 36 may be inserted into the instrument bracket 64 with a snap fit connection. The relatively rigid proximal portion 36 may be, e.g., approximately 5 to 6 inches long, and may be long enough to extend along the entire length of the instrument bracket 64 or a portion thereof. Providing the elongated member 34 with the relatively rigid proximal portion 36 and the relatively flexible distal portion 38 may be useful, for example, for controlling instruments 30 that include end effectors 32 since guiding the rigid proximal portion 36 with the instrument bracket 64 may assist with axial alignment of the instrument 30 with respect to the elongated member 20, and may allow longitudinal and rotational movement of the instrument 30. For example, guiding the rigid proximal portion 36 with the instrument bracket 64 may allow easier control of the movement of the end effector 32 of the instrument 30 since the rigid proximal portion 36 is less likely to be floppy or to bend when contacting the instrument bracket 64. Further, supporting the rigid proximal portion 36 in the instrument bracket 64 may allow the user to leave the instrument 30 in place within the instrument bracket 64 when the user releases the instrument 30.

Optionally, the relatively rigid proximal portion 36 may include markings, such as lines at set increments (e.g., longitudinally, rotationally, etc.), to allow the user to visually determine and gauge the longitudinal and rotational movement of the relatively rigid proximal portion 36 within the instrument bracket 64.

The distal portion 38 and the proximal portion 36 may be dimensioned to provide a stop that limits longitudinal movement of the instrument. For example, the proximal portion 36 may be dimensioned to be prevented from entering the port 24 of the elongated member 20. The distal portion 38 may have an approximately equal or smaller diameter (or width or other dimension) than the port 24, and the proximal portion 36 may have a larger diameter (or width or other dimension) than the port 24. Thus, when the instrument 30 is advanced distally into the elongated member 20, the distal portion 38 may be inserted through the port 24, but the proximal portion 36 may be prevented from entering the port 24, thereby forming a stop that limits the longitudinal movement of the instrument 30. If the length of the distal portion 38 and the distance between the port 24 and the distal end of the elongated member 20 are known, the stoppage of the longitudinal movement of the instrument 30 may indicate to the user, for example, a location of the end effector 20 in the patient, the extension of the end effector 20 with respect to the distal end of the elongated member 20, etc.

As shown in FIGS. 1, 3, and 4, the instrument 30 also includes a control device 40 located near the proximal end of the instrument 30. The control device 40 may be connected to the end effector 32 via the cables 46, 56 or other devices for connecting the control device 40 to the end effector 32. For example, in place of cables 46, 56, bare wires, insulated wires or cables, or other elongate flexible members may be used. The control device 40 allows the user to control the end effector 32, such as the articulation (e.g., orientation, position, movement, etc.) and functionality of the end effector 32. For example, the control device 40 may control the end effector 32 to move the end effector 32 longitudinally (e.g., proximally, distally, etc.), laterally (e.g., up, down, left, right, etc.), and/or rotationally (e.g., clockwise, counterclockwise, etc.). As indicated in FIG. 2, moving the end effector 32 laterally may include articulating or moving the end effector 32 up (U), down (D), left (L), and/or right (R), for example, with respect to an adjacent portion of the instrument 30. The control device 40 may also control the functionality of the end effector 32, such as by initiating a function or action of the end effector 32, e.g., opening, closing, etc.

The endoscopy system 10 may further include a frame 60 (FIG. 1) for supporting and positioning the elongated member 20 (such as an endoscope) and/or the instrument 30. The frame 60 may have a variety of configurations depending on patient location, spacing, ergonomics, physician preference, and/or the availability of an operating table space. As shown in FIG. 1, the frame 60 may include an elongated member bracket 62, the instrument bracket 64 described above, and/or an adjustable support 66. The elongated member bracket 62 may include a slot or surface, e.g., with generally U-shaped cross-section, to slidably receive and support the elongated member 20. The adjustable support 66 supports the elongated member 20 on the elongated member bracket 62, and may include a hinge, a telescoping section, or other device configured to modify the position (translational, rotational, etc.) of the elongated member 20. The frame 60 may also include an adjustment mechanism configured to adjust the position (translational, rotational, etc.) of the elongated member bracket 62 and the instrument bracket 64 with respect to each other, and/or may include locking mechanisms to lock the brackets 62, 64 and/or the support 66 in place and also to lock the devices within the corresponding brackets.

The exemplary control device 40 shown in FIGS. 1, 3, and 4 is configured to be manipulated by a user, or a mechanical or other type of controller. The control device 40 includes a ball and socket joint 42 and a handle portion 50 located near the proximal end of the instrument 30. The ball and socket joint 42 includes a ball 43 rotatably received within a socket 44. The ball and socket joint 42 connects the elongated member 34 of the instrument 30 to the handle portion 50 so that the handle portion 50 is configured to pivot with respect to the elongated member 34 of the instrument 30 via the rotation of the ball 43 in the socket 44.

The handle portion 50 may include a shaft 52 that is fixedly connected at one end to the ball 43 of the ball and socket joint 42. A ring 53 or other handle actuator may be formed on the opposite end of the shaft 52, and a movable member 54 (e.g., a spool, a ring, a lever, etc.) may be slidably disposed on the shaft 52 so that the movable member 54 is movable with respect to the ball and socket joint 42, the shaft 52, and the ring 53. For example, the movable member 54 may include a finger spool that is shaped to be gripped between two fingers, and the ring 53 may be shaped to receive the user's finger or thumb (a finger or thumb ring). As a result, the user may be able to grip the movable member 54 and the ring 53 with a single hand to move the movable member 54 with respect to the ring 53 and to pivot the handle portion 50 with respect to the ball and socket joint 42.

Alternatively, more than one movable member 54 may be provided on the shaft 52 to provide a variety of controls. For example, a first movable member 54 (e.g., a spool, a ring, a lever, etc.) may be provided to control up/down movement, a second movable member 54 (e.g., a spool, a ring, a lever, etc.) may be provided to control left/right movement, and/or a third movable member 54 (e.g., a spool, a ring, a lever, etc.) may be provided to actuate the end effector 32.

Alternatively, the handle portion 50 may include another type of grip for handling by the user. For example, the handle portion 50 may include a scissor-type grip, pistol grip, two-finger loop, or thumb stroke loop. FIG. 6 shows an alternative exemplary embodiment in which the ring 53 is a thumb stroke loop 70 (or thumb ring) shaped to receive the user's thumb (or finger) and the movable member 54 includes one or more finger loops 72. In the illustrated embodiment, two finger loops 72 are provided. As a result, the user may be able to grip the thumb stroke loop 70 and the two finger loops 72 with the thumb and two fingers of a single hand to move the movable member 54 (e.g., the two finger loops 72) with respect to the thumb stroke loop 70 and to pivot the handle portion 50 with respect to the ball and socket joint 42.

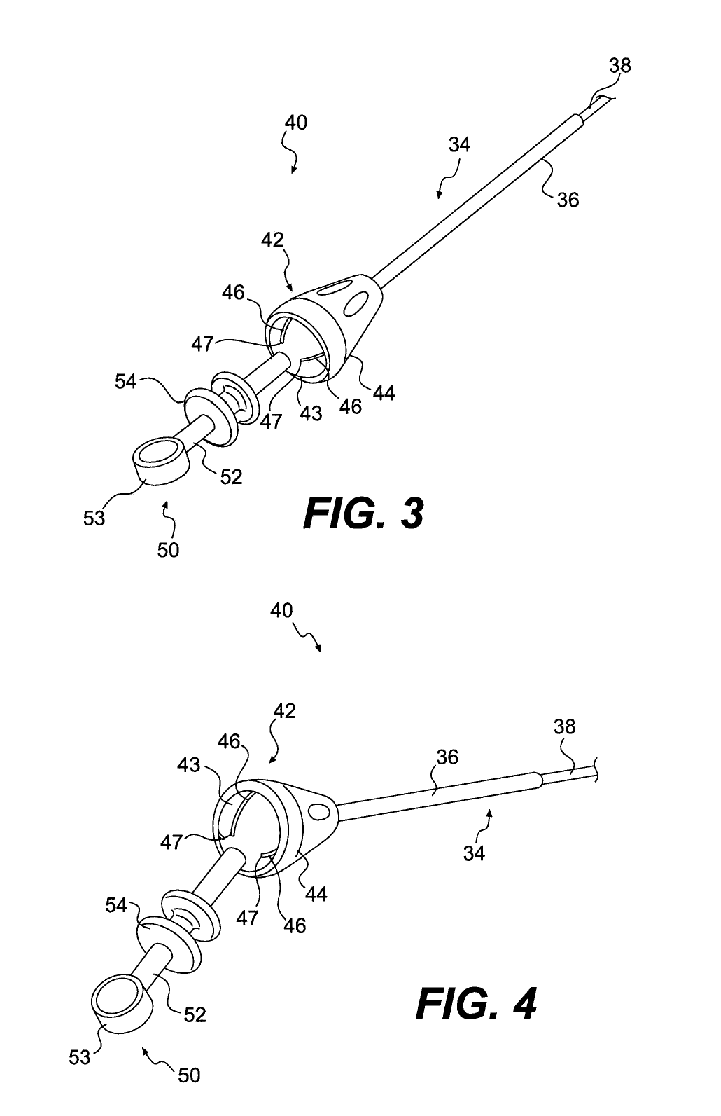

One or more articulation control cables 46 may be fixedly connected at one end 47 to the ball 43. For example, in exemplary embodiments, two, three, or four articulation control cables 46 may be fixedly connected at their respective ends 47 to the ball 43. The articulation control cables 46 may extend through an opening in the socket 44 and through the elongated member 34 of the instrument 30, and an opposite end of the articulation control cables 46 may connect to the end effector 32. For example, as shown in FIGS. 3 and 4, the end 47 of each articulation control cable 46 may be affixed directly to an outer surface of the ball 43, e.g., near a proximal side of the ball 43, and/or near a connection between the shaft 52 and the ball 43. The outer surface of the ball 43 may include grooves to receive the articulation control cables 46.

The articulation control cables 46 connect the ball 43 to the end effector 32 to control the articulation of the end effector 32. For example, rotating the ball 43 within the socket 44 using the handle portion 50 (e.g., by moving the ring 53) causes the ends 47 of the articulation control cables 46 connected to the ball 43 to also rotate, thereby pulling one or more of the articulation control cables 46 at least partially around the ball 43. As a result, tension may increase in the articulation control cable(s) 46 that are pulled around the ball 43, which may cause the end effector 32 to move laterally. This allows the user to control one or more degrees of freedom, e.g., up/down and/or left/right movement, of the end effector 32 with one or both hands, as desired.

The number and placement (e.g., the location of the connection of the ends 47 to the ball 43) of the articulation control cables 46 may vary depending, for example, on the desired number of degrees of freedom for controlling the end effector 32, the desired accuracy of the movement of the end effector 32, etc. For example, in the embodiment shown in FIGS. 3 and 4, four articulation control cables 46 are provided (separated from each other by approximately 90 degrees with respect to an axis extending through the ball 43 and the shaft 52), and the articulation control cables 46 are capable of providing at least two degrees of freedom, e.g., up/down and/or left/right movement. Alternatively, two articulation control cables 46 may be provided (separated from each other by approximately 90 degrees with respect to the axis extending through the ball 43 and the shaft 52) to provide at least two degrees of freedom, e.g., up/down and/or left/right movement.

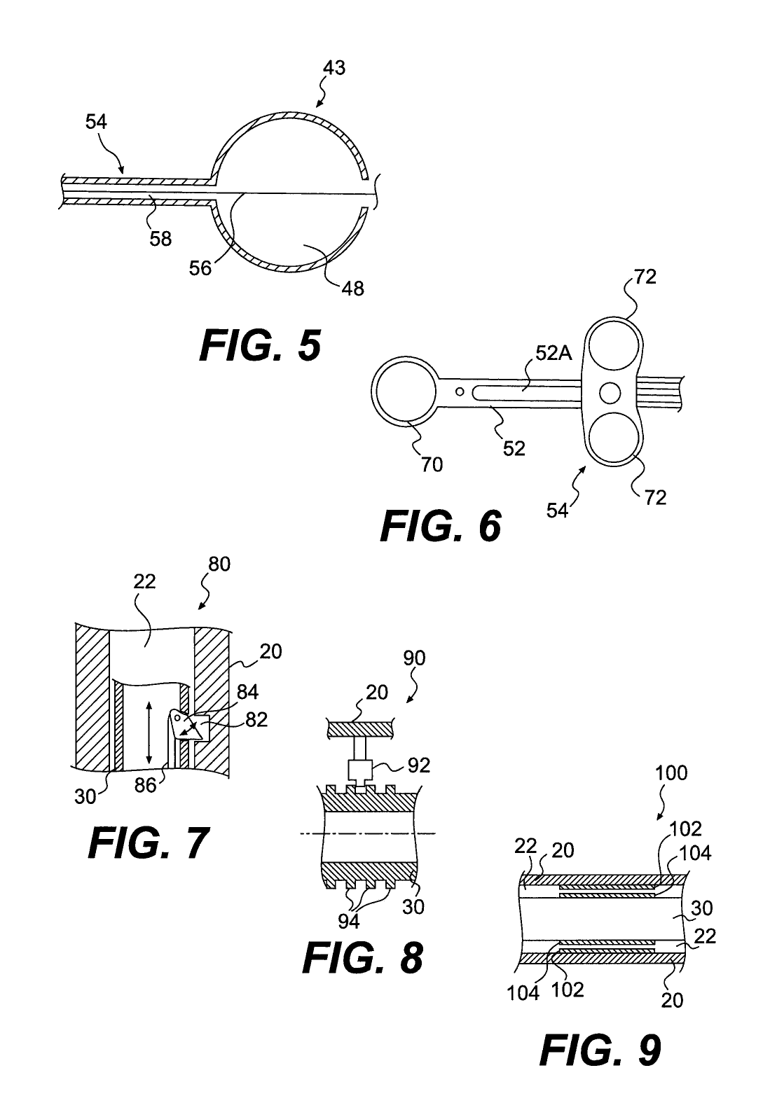

Another control cable 56 (FIG. 2) may be connected to an inner surface (not shown) of the movable member 54, and the control cable 56 may extend through a slot 52a (FIG. 6) in the shaft 52. Then, as shown in FIG. 5, the control cable 56 may extend through a channel 58 extending axially through the shaft 52 and through a cavity 48 or channel in the ball 43. The control cable 56 may also extend through the socket 44 and then through the elongated member 34 of the instrument 30, as shown in FIG. 2. The end of the control cable 56 may connect to the end effector 32.

The control cable 56 may connect the movable member 54 to the end effector 32 to control the functionality of the end effector 32. For example, when the user pulls the movable member 54 towards the ring 53 (proximally), the movable member 54 may pull the control cable 56. As a result, tension may increase in the control cable 56, which may cause the end effector 32 to operate or perform a function, e.g., opening or closing a pair of jaws, or initiating another type of action. Moving the movable member 54 in the opposite direction away from the ring 53 (distally) may release the tension in the control cable 56, thereby performing another function of the end effector 32, e.g., closing or opening the pair of jaws, or stopping the action that was initiated by pulling the movable member 54. Alternatively, more than one control cable 56 may be provided, e.g., to control more than one end effector 32 of the instrument 30.

In addition, the control device 40 may be capable of controlling longitudinal movement of the end effector 32. For example, the user may pull or push the instrument 30 using the ring 53. The ring 53 is located on the shaft 52, which is connected to the ball and socket joint 42, which is in turn connected to the elongated member 34 of the instrument 30. Thus, moving the ring 53 longitudinally may cause the instrument 30 to move longitudinally with respect to the frame 60 supporting the elongated member 20 and/or the instrument 30 (e.g., the instrument bracket 64), which in turn may cause the end effector 32 to also move longitudinally with respect to the frame 60 supporting the elongated member 20 and/or the instrument 30.

The control device 40 may also be capable of controlling rotational movement of the end effector 32. For example, the user may rotate the elongated member 34 of the instrument 30, which may rotate the end effector 32. For example, the user may grip the socket 44, which is connected to the elongated member 34 of the instrument 30, to rotate the end effector 32. Thus, the instrument 30 may be rotated with respect to the frame 60 supporting the elongated member 20 and/or the instrument 30 (e.g., the instrument bracket 64), which in turn may cause the end effector 32 to rotate. Alternatively, or in addition, rotation of the instrument 30 (and therefore the end effector 32) may be caused by rotating the ball 43 in the socket 44, rotating the ring 53, rotating the movable member 54, etc.

As a result, the user may control the endoscopy system 10 more easily and efficiently. For example, the user may be able to manipulate at least one degree of freedom of the instrument 30 using the control device 40, and the control device 40 may be manipulated with a single hand. Alternatively, the control device 40 may control at least two, three, or four degrees of freedom.

In the exemplary embodiments described above and shown in FIGS. 1, 3, and 4, the ball 43 is attached to the shaft 52 of the handle portion 50 and the socket 44 is attached to the elongated member 34 of the instrument 30. Alternatively, the ball 43 and the socket 44 may be interchanged such that the socket 44 is attached to the shaft 52 of the handle portion 50 and the ball 43 is attached to the elongated member 34 of the instrument 30. In such an embodiment, the socket 44 may be rotated with respect to the ball 43, and the articulation control cables 46 may be affixed to the inner surface of the socket 44 (instead of the outer surface of the ball 43).

In the illustrated embodiment of FIGS. 1, 3, 4, and 5, the ball 43 is generally spherical. Alternatively, the ball 43 may be a portion of a spherical surface, e.g., greater than half of a spherical surface, hemispherical, semispherical, less than half of a spherical surface, a spherical cap, etc. As another alternative, the ball 43 may be a surface with a curvature, but may not necessarily be spherical or a portion of a spherical surface. The corresponding socket 44 may be shaped to receive the ball 43 and therefore may have a curvature, but may not necessarily be spherical. Accordingly, the ball 43 and the socket 44 may be any combination of interlocking, curved surfaces.

The instrument 30 described above may be used for various different types of elongated members 20, including various endoscopes, guide tubes, catheters, or any other medical devices inserted into the body through any anatomic opening. The instrument 30 may be universally adapted for use with elongated members 20 having different configurations, such as different sizes, features, etc., which may be more cost efficient. Multiple instruments 30 may be inserted simultaneously through the elongated member 20. The instrument(s) 30 may each include one or more end effectors 32.

The control device 40 described above allows the user to control the movement and operation of the instrument 30 with a single hand, which may free the user's other hand to control other devices or instruments. The control device 40, including the ball and socket joint 42 may be simple to use, ergonomic, easier to control, and universally adapted for use with different end effectors 32 and instruments 30. The control device 40 may also provide an intuitive user interface that may be easy to handle and activate. The control device 40 may also provide a simple design that permits the articulation and other movement of the end effector 32.

Providing both the relatively rigid proximal portion 36 and the relatively flexible distal portion 38 may allow for greater and more precise control using the control device 40. Furthermore, providing the relatively rigid proximal portion 36 in the instrument bracket 64 allows for a more ergonomic design, single-handed control and operation of the control device 40, greater ease of use, and greater stability of the control device 40 within the endoscopy system 10. For example, the instrument bracket 64 is capable of guiding and serving as a bearing surface for the relatively rigid proximal portion 36, which is capable of sliding within the instrument bracket 64. The instrument bracket 64 is also capable of holding the relatively rigid proximal portion 36 at a precise location and/or locking the relatively rigid proximal portion 36 in place (e.g., longitudinally, laterally, rotationally, etc.). The instrument bracket 64 also allows the user to move the relatively rigid proximal portion 36, e.g., longitudinally and/or rotationally, with respect to the instrument bracket 64.

The endoscopy system 10 may include one or more braking mechanisms, locking mechanisms, or other devices configured to hold the instrument 30 (e.g., the proximal portion 36, the distal portion 38, etc.) in place (e.g., within the instrument bracket 64, the port 24, any portion of the elongated member 20, etc.). Such a device may provide a locking or non-locking connection to hold the instrument 30 in place at least temporarily, e.g., until the user moves the instrument 30 or releases the instrument 30 from a locked position.

For example, FIGS. 7-9 show exemplary locking mechanisms 80, 90 and an exemplary braking mechanism 100 for positioning the instrument 30 with respect to the elongated member 20. Similar locking and/or braking mechanisms may also be included to position the instrument 30 with respect to the instrument bracket 64. The portion of the elongated member 20 shown in FIGS. 7-9 may be any portion of the elongated member 20, such as the a distal portion of the elongated member 20, a proximal portion of the elongated member 20, a central portion of the elongated member 20 between the proximal and distal portions, the port 24, etc. The portion of the instrument 30 shown in FIGS. 7-9 may be any portion of the instrument 30, such as any portion of the proximal portion 36, the distal portion 38, etc. Alternatively, the endoscopy system 10 may include multiple locking mechanisms 80, 90 and/or braking mechanisms 100, e.g., in various locations along the elongated member 20 and/or the instrument bracket 64 and/or various locations along the proximal portion 36 and the distal portion 38 of the instrument 30.

In the embodiment shown in FIG. 7, the inner surface of the elongated member 20 may include a groove 82, and an outer surface of the instrument 30 that is configured to face the inner surface of the elongated member 20 may include a pivotable latch 84. When the instrument 30 moves distally within the elongated member 20, the pivotable latch 84 may slide against the inner surface of elongated member 20 without catching the groove 82. Thus, the instrument 30 does not lock in place with respect to the elongated member 20. When the instrument 30 moves proximally within the elongated member 20, the pivotable latch 84 may be configured to catch the groove 82 and prevent the pivotable latch 84 from moving proximal to the groove 82. Accordingly, the locking mechanism 80 may prevent the user from pulling the instrument 30 completely out of the elongated member 20. The pivotable latch 84 may be connected to a control cable 86 that extends to the handle portion 50 of the instrument 30, and the user may pull the control cable 86 to rotate the pivotable latch 84 to release the pivotable latch 84 from the groove 82, thereby releasing the locking mechanism 80 and allowing the user to pull the instrument 30 proximally with respect to the elongated member 20.

In the embodiment shown in FIG. 8, the elongated member 20 may include an extendable member 92 that may be controlled to extend radially inward or retract radially outward within the channel 22 in the elongated member 20. For example, the extendable member 92 may be biased to extend radially inward until the user presses a button to release the extendable member 92 to retract the extendable member 92 radially outward. Alternatively, the extendable member 92 may be a screw that is rotatable in one direction to extend radially inward and rotatable in the opposite direction to retract radially outward. As another alternative, the extendable member 92 may be electronically driven. The outer surface of the instrument 30 may include a plurality of teeth 94 that are arranged in series along the length of the instrument 30. When the instrument 30 is in the elongated member 20, the user may extend the extendable member 92 radially inward to engage a slot between two adjacent teeth 94 on the instrument 30, thereby locking the instrument 30 in place within the elongated member 20. When the user wants to move the instrument 30 again, the user may retract the extendable member 92 from the slot between the teeth 94. Accordingly, the locking mechanism 90 allows the user to set the position of the instrument 30 within the elongated member 20 and to assist in preventing inadvertent movement of the instrument 30 with respect to the elongated member 20.

In the embodiment shown in FIG. 9, the inner surface of the elongated member 20 may include a friction surface 102 or other engaging device capable of forming a friction fit with at least one friction surface 104 or other engaging device of the instrument 30 in order to at least temporarily hold the instrument 30 in place as the friction surfaces 102, 104 contact or slide against each other. The respective surfaces 102, 104 may be formed of, for example, silicone to form a silicone-to-silicone interface capable of forming a friction fit between the instrument 30 and the instrument bracket 64, the port 24, or a portion of the elongated member 20. In the illustrated embodiment, the friction surfaces 102, 104 extend radially inward or outward from the respective surfaces of the elongated member 20 and the instrument 30 with a gap therebetween. Alternatively, the friction surfaces 102, 104 may be flush with the respective surfaces of the elongated member 20 and the instrument 30. As another alternative, substantially no gap may be provided between the friction surfaces 102, 104. The lengths of the friction surfaces 102, 104 may vary with respect to each other, to the elongated member 20, to the instrument 30, etc., depending on the desired amount of braking provided by the braking mechanism 100, etc. Accordingly, as the instrument 30 is articulated, advanced/retracted, rotated, or actuated, the user may release or let go of the instrument 30, and the braking mechanism 100 may hold the instrument 30 in place (e.g., longitudinally, laterally, rotationally, etc.) and in the last position in which the instrument 30 was held when the user released the instrument 30.

The user may use one or more of the locking mechanisms 80, 90 and/or braking mechanism 100 to lock or position the instrument 30 in place (e.g., longitudinally, laterally, rotationally, etc.). As a result, the locking mechanisms 80, 90 and/or braking mechanism 100 may be used to fix, lock, or position the instrument 30 relative to the elongated member 20, which may free the user's hand to control other devices or instruments and which may reduce hand fatigue while keeping the instrument 30 ready for use. The locking mechanisms 80, 90 and/or braking mechanism 100 may also assist in preventing the removal of the instruments 30 from the sterile site and/or preventing the instruments 30 from falling out of the elongated member 20.

In an alternative embodiment, the instrument 30 may include one or more biasing devices capable of returning the instrument 30 to a normal or desired position or orientation. For example, the articulation control cables 46, the control cable 56, the ball and socket joint 42, and/or other portions of the instrument 30 may be formed of an elastic material configured to extend/contract, bend, twist, etc., when the user applies a certain force on the instrument 30, but may return to a normal or desired position when the force is removed. Alternatively, springs or other biasing devices (not shown) may be provided, e.g., around the elongated member 34, attached to the ball and socket joint 42, etc. For example, the spring may be connected between the instrument 30 and the instrument bracket 64, the port 24, or a portion of the elongated member 20. As the user articulates, advances/retracts, rotates, or actuates the instrument 30, the instrument 30 may move away from a normal or desired position and the spring may extend, retract, twist, etc. When the user releases the instrument 30, the spring may cause the instrument 30 to return to its normal or desired position.

In an alternative embodiment, the instrument 30 may include at least one key or projection (not shown) extending outwardly from an outer surface of, e.g., the elongated member 34, the ball and socket joint 42, etc. The key may be shaped to be received within a corresponding keyway, opening, or slot (not shown) in an inner surface of the instrument bracket 64, the port 24, and/or the elongated member 20. Alternatively, the instrument 30 may include at least one keyway, opening, or slot on the outer surface, and the inner surface of the instrument bracket 64, the port 24, and/or the elongated member 20 may include the corresponding key or projection. As a result, the instrument 30 may be keyed to the instrument bracket 64, the port 24, and/or the elongated member 20 and may allow the user to more precisely control the articulation, advancement, retraction, rotation, or actuation of the elongated member 34 and/or the end effector 32 of the instrument 30, such as the transmission of a torque.

In an alternative embodiment, the ball and socket joint 42 may include a relief spring (not shown) or other biasing device, e.g., disposed within the socket 44. The relief spring may allow the user to adjustably lock the orientation (e.g., rotation) of the ball 43 in place with respect to the socket 44. For example, the relief spring may apply a biasing force that pushes the ball 43 against the socket 44 to lock the ball 43 in place in the socket 44. The user may push on an exposed surface of the ball 43 to provide a force that counters the biasing force of the spring, thereby causing the ball 43 to move away from the socket 44. While the user pushes the ball 43 away from the socket 44, the user may also move the ball 43 rotationally within the socket 44 to adjust the position of the ball 43 with respect to the socket 44. When the ball 43 is at a new desired position, the user may release the ball 43, thereby causing the relief spring to apply the biasing force that pushes the ball 43 back against the socket 44, thereby locking the ball 43 in place at the desired position selected by the user. As a result, the relief spring may secure the ball 43 in place with respect to the socket 44 while still allowing the user to adjust the position of the ball 43 with respect to the socket 44.

In an alternative embodiment, the ball and socket joint 42 may be attached to the proximal end of the elongated member 20. The socket 44 may be attached to the proximal end of the elongated member 20, and the ball 43 and/or the socket 44 may include a port (not shown) for receiving one or more instruments 30. The port may also include a seal for preventing inflow and/or outflow of fluids from and/or to the patient's body via the channel 22. For example, the handle portion 50 (e.g., the shaft 52, the ring 53, the movable member 54, etc.) shown in FIGS. 1, 3, 4, and 5 may be replaced with the port and the seal in the ball 43. A user may insert one or more instruments 30 through the seal and the port, and then advance the instrument(s) 30 through the channel 22 in the elongated member 20 and into the patient so that the instrument(s) 30 may be used during an endoscopic procedure.

The various components of the endoscopy system 10 described herein may be made of a suitable biocompatible material and may be flexible, for example, to traverse tortuous anatomy in the body. Any aspect set forth in any embodiment may be used with any other embodiment set forth herein. Every device and apparatus set forth herein may be used in any suitable medical procedure, may be advanced through any suitable body lumen and body cavity, and may be used to visualize, acquire, or remove tissue from any suitable body portion.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and their equivalents.

What is claimed is:

1. An instrument comprising:
an end effector located near a distal end of the instrument;
an elongated member connected to the end effector;
a handle located near a proximal end of the instrument;
a ball and socket joint including a ball rotatable in a socket, the ball and socket joint connecting the elongated member to the handle, the handle being configured to move with respect to the elongated member via the ball and socket joint; and
a control member connected to one of the ball or the socket, the control member extending through the elongated member and connecting the ball and socket joint to the end effector to control movement of the end effector.

2. The instrument of claim 1, wherein the handle is configured to move with respect to the elongated member to control a lateral movement of the end effector.

3. The instrument of claim 1, wherein the control member is connected to a surface of the one of the ball or the socket, the surface including a surface of the ball that contacts the socket or a surface of the socket that contacts the ball.

4. The instrument of claim 1, wherein the control member is connected to a surface of the ball and extends through an opening in the socket.

5. The instrument of claim 1, wherein the ball is rotatable in the socket to adjust a tension of the control member, and the tension of the control member is adjustable to control the movement of the end effector.

6. The instrument of claim 1, wherein the control member is one of a plurality of control members connected to the one of the ball or the socket to connect the ball and socket joint to the end effector to control movement of the end effector.

7. The instrument of claim 1, wherein:
the elongated member is a first elongated member; and
the first elongated member of the instrument is configured to be at least partially inserted through a channel extending longitudinally within a second elongated member, with a distal end of the second elongated member being configured to be inserted into a patient.

8. The instrument of claim 7, wherein the first elongated member is movable with respect to a frame supporting the first elongated member and the second elongated member, to control a longitudinal movement of the end effector.

9. The instrument of claim 7, wherein the first elongated member includes a flexible portion configured to be slidably inserted into the channel in the second elongated member.

10. The instrument of claim 9, wherein the first elongated member includes a rigid proximal portion configured to be slidably inserted into a bracket attached to a frame supporting the first elongated member and the second elongated member.

11. An instrument comprising:
an end effector located near a distal end of the instrument;
an elongated member connected to the end effector;
a handle located near a proximal end of the instrument, the handle including a shaft and a movable member that is movable with respect to the shaft;
a ball and socket joint connecting the elongated member to the handle, the handle being configured to move with respect to the elongated member via the ball and socket joint; and
a control member connected to the movable member and the end effector, the control member extending through the ball and socket joint and the elongated member, the movable member being configured to move with respect to the shaft to control the end effector via the control member.

12. The instrument of claim 11, wherein the shaft is connected to at least one of a ball or a socket of the ball and socket joint.

13. The instrument of claim 11, wherein the moveable member is movable with respect to the shaft to adjust a tension of the control member to control the end effector.

14. The instrument of claim 11, wherein the moveable member is movable with respect to the shaft to open or close the end effector.

15. The instrument of claim 11, wherein the movable member includes a finger spool.

16. The instrument of claim 11, wherein:
the control member is a first control member; and
the instrument further includes a second control member connected to one of a ball or a socket of the ball and socket joint, the second control member extending through the elongated member and connecting the ball and socket joint to the end effector to control movement of the end effector.

17. The instrument of claim 11, wherein:
the elongated member is a first elongated member; and
the first elongated member of the instrument is configured to be at least partially inserted through a channel extending longitudinally within a second elongated member, with a distal end of the second elongated member being configured to be inserted into a patient.

18. A method for controlling an instrument, the method comprising:
controlling an end effector near a distal end of an instrument by:
pivoting a handle with respect to an elongated member of the instrument via a ball and socket joint of the instrument to control a first control member connecting the ball and socket joint to the end effector, the first control member being connected to one of a ball or a socket of the ball and socket joint; and
sliding a movable member of the handle of the instrument with respect to a shaft of the handle to control a second control member connecting the movable member to the end effector.

19. The method of claim 18, wherein:
the first control member extends through the elongated member; and
the second control member extends through the ball and socket joint and the elongated member.

20. The method of claim 18, further comprising adjusting a position of the end effector by rotating the ball in the socket.
